(19) United States
(12) Patent Application Publication
(10) Pub. No.: US 2023/0226130 A1
(43) Pub. Date: Jul. 20, 2023

(54) EXTRACTION TECHNIQUES TO PRESERVE CANNABINOID AND TERPENOID PROFILES

(71) Applicant: CReDO Science, LLC, Austin, TX (US)

(72) Inventor: Ethan Russo, Vashon, WA (US)

(21) Appl. No.: 18/000,567

(22) PCT Filed: Jun. 3, 2021
(86) PCT No.: PCT/US2021/035794

§ 371 Date: Dec. 2, 2022

Related U.S. Application Data
(60) Provisional application No. 63/034,823, filed on Jun. 4, 2020.

(51) Int. Cl. A61K 36/185

(52) U.S. Cl. CPC A61K 36/185; A61K 2236/15; A61K 2236/37; A61K 2236/333; A61K 2236/31

(57) ABSTRACT

The invention generally relates to methods and systems for preparing a plant material for extraction of useful cannabinoids and terpenoids from Cannabis.

### CLAIM OF PRIORITY UNDER 35 U.S.C. § 119

The present Application for Patent claims priority to Provisional Application No. 63/034,823 entitled “EXTRACTION TECHNIQUES TO PRESERVE CANNABINOIDS AND TERPENOID PROFILES” filed Jun. 4, 2020 and assigned to the assignee hereof and hereby expressly incorporated by reference herein.

### BACKGROUND

### Field

This invention provides methods and systems for preparing a Cannabis plant material for extraction and method and systems for extraction of a compound from said plant material.

### Background

Phytocannabinoids and cannabis terpenoids are the primary medicinal components for most cannabis-based medicines. Current extraction methods generally operate under the assumption that the material would be smoked. Cannabis is commonly dried and cured prior to use, either in the sun (e.g., in Rif Mountains, Morocco with unmanicured cannabis), or under controlled humidity conditions (e.g., at GW Pharmaceuticals). Studies have shown that there are considerable losses of monoterpenoids during drying and curing, which can range from 31-55.2%, depending on the length of the process (Ross, S. A., & ElSohly, M. A. (1996). The volatile oil composition of fresh and air-dried buds of Cannabis sativa. J Nat Prod, 59(1), 49-51). The original intent of this process was to improve “smoke-ability” by oxidation of chlorophyll to phytol, and to reduce chances of mold. However, in the process, the “headspace volatiles” of cannabis, the lower molecular weight monoterpenoids, are squandered. Therefore, such extraction methods may be counterproductive because of the reduced extraction of useful compounds and/or the inclusion of chlorophyll and many other pharmacologically unnecessary compounds.

### SUMMARY

Some embodiments of the invention relate to a method of preparing a Cannabis plant material for extraction of a compound. In some embodiments, the method can include the steps of: (a) obtaining fresh plant material; (b) flash freezing the plant material in a container containing a cooling material for a period of time; and/or (c) dry sifting the plant material in a cold environment to isolate trichomes. In some embodiments, the isolated trichomes can include a biochemical profile of cannabinoids or terpenoids that is substantially similar to the biochemical profile of the fresh plant material.

In some embodiments, the cooling material is dry ice.

In some embodiments, the time between step (a) and (b) is less than 5 minutes.

In some embodiments, the period of time in step b is at least 10 minutes.

In some embodiments, the dry sifting step can include placing the frozen plant material in a rotating drum with perforations.

In some embodiments, the isolated trichomes can be collected in a tray underneath the drum.

In some embodiments, no solvents are used in the method.

In some embodiments, the relative yield of isolated trichomes can be at least 2× greater than a yield of isolated trichomes from a comparison process, wherein the comparison process begins with an equivalent amount of starting material and comprises drying the plant material prior to isolation of trichomes.

In some embodiments, the relative yield of isolated trichomes can be at least 3× greater than the yield of isolated trichomes from the comparison process.

In some embodiments, the relative yield of isolated trichomes can be at least 4× greater than the yield of isolated trichomes from the comparison process.

Some embodiments of the invention relate to a system for preparing a Cannabis plant material for extraction of a compound using a method described herein. In some embodiments, the system can include a first container containing contents comprising a cooling material adapted to cool a starting material to a temperature of less than −10° C.; a second container containing contents comprising the first container; and/or a dry-sifting device comprising a housing, a drum and a tray.

Some embodiments of the invention relate to a method of extracting a compound from a Cannabis plant using a method described herein and further including extracting compounds from trichomes and optionally, decarboxylating the compounds. In some embodiments, the isolated compound can include at least 0.5× more purity compared to fresh flower, wherein the purity is defined by percent total cannabinoids and terpenoids of the total mass.

In some embodiments, the extraction step can include use of an extraction medium or process selected from at least one of supercritical CO2, cold ethanol, and steam distillation.

Some embodiments of the invention relate to a lipid nanoparticle preparation produced from an isolated trichome produced by any of the methods disclosed herein.

### BRIEF DESCRIPTION OF THE DRAWINGS

FIG. 1 is a diagram of a fresh-flower dry-ice kief (FDK) dry sifting device used in an embodiment of the invention.

FIG. 2 depicts photographs from the experiment conducted in Example 2. FIG. 2a is a photograph of an FDK sample. FIG. 2b is a photograph of Dried Kief sample. Note leaf and other extraneous matter in the dried kief sample.

FIG. 3 depicts photographs from the experiment conducted in Example 3. FIG. 3a is a photograph of an FDK sample. FIG. 3b is a photograph of Dried Kief sample. Note leaf and other extraneous matter in the dried kief.

FIG. 4 depicts photographs from the experiment conducted in Example 4. FIG. 4a is a photograph of an FDK sample. FIG. 4b is a photograph of Dried Kief sample. Note the FDK sample looks much purer, with rare leaf flecks in comparison to dried kief.

FIG. 5 depicts photographs from the experiment conducted in Example 5. FIG. 5a is a photograph of an FDK sample. FIG. 5b is a photograph of Dried Kief sample. Note the FDK sample looks much purer, with rare leaf flecks in comparison to dried kief.

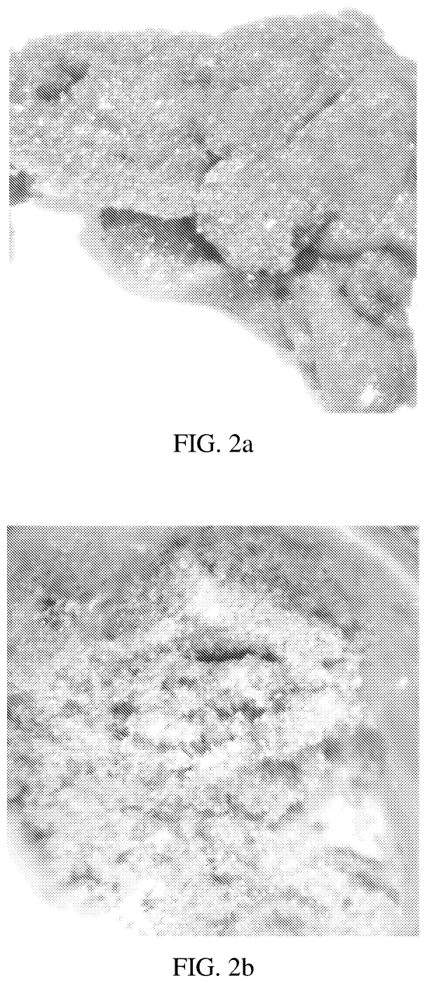

FIGS. 2-5 are available in color online at https://credo-science<dot>com <slash>kryokief-pics.

### DETAILED DESCRIPTION

The present invention relates to novel extraction and processing methods and systems for Cannabis plants. Phytocannabinoids and cannabis terpenoids are produced in greatest abundance in the capitate glandular trichomes of unfertilized female inflorescences. Most other material in the flowers, leaves and other plant parts of Cannabis are extraneous to the majority of Cannabis medicine preparations. The methods disclosed herein relate to the extraction of useful phytocannabinoids and Cannabis terpenoids from trichomes with minimal inclusion of extraneous compounds. The methods disclosed herein also relate to preserving, in extracted products, the biochemical profile of fresh flowers.

The present invention relates to the extraction of key active pharmaceutical ingredients (APIs) from a Cannabis plant. Such APIs can include various phytocannabinoids and terpenoids which are secreted into and contained within the trichome envelope. Such APIs can include the phytocannabinoids and terpenoids disclosed in the book chapter by Ethan B. Russo and Jahan Marcu, “Cannabis Pharmacology: The Usual Suspects and a Few Promising Leads.” In: David Kendall and Stephen P. H. Alexander, editors, Advances in Pharmacology, Vol. 80, Burlington: Academic Press, 2017, pp. 67-134; and Russo, E. B. (2011). Taming THC: potential cannabis synergy and phytocannabinoid-terpenoid entourage effects. Br J Pharmacol, 163(7), 1344-1364. doi:10.1111/j.1476-5381.2011.01238.x; the entire contents of each of the foregoing are fully incorporated by reference herein.

Embodiments of the invention relate to extracting capitate glandular trichomes from fresh flowers while preserving the native biochemical profile of phytocannabinoids and cannabis terpenoids. The inventors have demonstrated the superiority of fresh frozen flowers as a feedstock for high potency, high purity cannabis trichome extracts without extraneous materials. Some embodiments of the invention relate to “fresh-flower, dry ice kief” (referred to herein as FDK) extraction which can preserve monoterpenoids and other APIs in a manner that can be more therapeutic in clinical practice. The term “dry ice” as used herein is not intended to limit the invention to dry ice. Dry ice can be used as a cooling mechanism in the invention and can be replaced, or used in addition to any other cooling mechanism capable of achieving the desired temperature or range of temperatures, such as use of a blast chiller or the like.

In some embodiments of the invention, Cannabis plant material is obtained. The plant material can include Cannabis inflorescence or flower. In some embodiments, fresh flowers are collected from a field or from a controlled indoor grow facility. “Fresh” can be defined as still being in a growing condition. In some embodiments, the method includes obtaining a fresh plant material. A non-limiting example of obtaining the fresh plant material can include cutting the flower from a live plant.

### Flash Freezing

In some embodiments, the plant material is immediately preserved in a cold environment. “Immediately” can be about 1, 2, 3, 4, 5, 10, 15, 30, 45, or 60 min after collecting the flower as described above. The cold environment can be a temperature of about less than −150° C., −125° C., −120° C., −100° C., −75° C., −50° C., −45° C. or −40° C. or less. The cold environment can be, for example, a container containing dry ice (solid CO₂) or any cooling material with surface temperature of about −90° C. to −70° C., or about −78.5° C. The plant material can be kept in the container for at least 15, 30, or 60 or more minutes. In some embodiments, the plant material can be kept in the container for 1 hr, 4 hrs, 8 hrs, 12 hrs, 24 hrs, 36 hrs, or 48 hours or more before processing. The plant material can be in direct contact with the dry ice or can be stored in a container with dry ice or both. The container can be any container capable of holding the plant material and/or the dry ice, such as a tube, bag, box, tray, or the like. In some embodiments, the plant material can be in direct contact with the dry ice in a first container, and the first container with the plant material and dry ice can be in a second container. The first or second container can be insulated with a material comprising insulating properties such as polyethylene. In some embodiments, the second container contains a third container wherein the third container sits above the first container. The third container can include dry ice. The second container can include openings for vapor egress. The openings can be holes, slits, ports, plugs or the like. In some embodiments, this process freeze-dries the plant material as its water content is sublimated and dehumidified by the vapor, which is allowed to escape. For example, when the openings of the second container are opened, egress of CO₂ and water vapor can be allowed, which permits laminar flow of vapor over and through plant material to maximize penetration and lyophilization effects. The lyophilized flowers can be stored subsequently in an industrial freezer prior to processing. This step produces a frozen plant material for processing. This step can be used in combination with drying, freeze-drying, lyophilization and/or the like.

### Dry Sifting

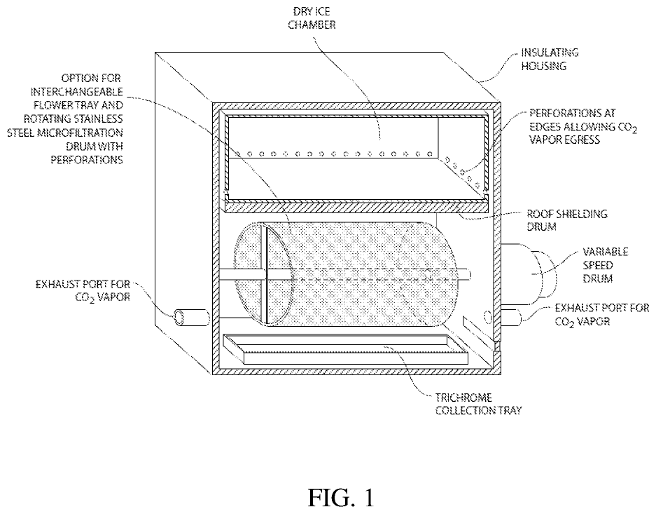

In some embodiments, the method or system includes processing the frozen plant material. The processing step can include dry sifting the frozen plant material or any other method that separates the trichomes from the rest of the plant material. Dry sifting can be done using a processing device/machine. FIG. 1 depicts the device in some embodiments. In some embodiments, the machine can include a housing with a chamber, a drum and a collecting tray. The chamber can hold dry ice or other cooling material such that the processing step is kept at a cold temperature. The cold temperature can be about −30° C., −25° C., −20° C., −18° C., −15° C., −10° C. In some embodiments, the temperature is kept at about −18° C. In some embodiments, the drum can be attached to a motor and can rotate. In some embodiments, the housing is made from a material such as polyethylene or stainless steel or the like. In some embodiments, the housing is insulated with urethane foam or the like. In some embodiments, the device/machine can process about 100, 150, 250, 500, 750, 1000, 1500, 2000, 2500, 3000, 3500, 4000, 4500, or 5000 or more grams of plant material at a time. In other embodiments, the device/machine can be much larger to achieve higher processing throughput. The drum can have drum perforations of a diameter of about 50, 100, 150, 200, 250μ, or more. The drum can rotate at 10, 15, 20, 25, 30, 33, 35, 40, 45, 50, 55, 60, 65, 70, 75, or more rpm. In some embodiments, the machine can be a machine such as the Pollinator® (see https://pollinator.nl/category/pollinator/) at a temperature of below −18° C. Dry sifting is a process that separates the trichomes from the plant material. The trichomes can be collected in the tray. The collected trichomes can be referred to as kief, hashish, or “enriched trichome preparation”, isolated trichomes (further information can be found in Potter, D. J. (2009). The propagation, characterisation and optimisation of Cannabis sativa L. as a phytopharmaceutical. (PhD). King's College, London, which is hereby fully incorporated by reference herein). In some embodiments, the processing step does not involve solvents. Dry sifting of the plant material can occur for 1 min, 2, min, 5 min, 10min, 15 min, 20 min, or more. Dry sifting of the plant material can occur until the plant material comprises less than 25%, 20%, 15%, 10%, 5% or less moisture content. After this step, the trichomes can be substantially free of extraneous material. Extraneous material can include leaves, stems, non-glandular trichomes devoid of useful phytochemicals, and the like. Substantially free can be defined as a reduction of at least 50%, 60%, 70%, 80%, 90%, 95%, 99%, or more of the extraneous material that was present prior to the dry sifting step.

In some embodiments, additional sieving steps or electrostatic techniques can be used to remove other plant debris, smaller trichomes, dust, other positively charged materials, and/or the like.

### Isolated Material for Extraction

The material obtained by the methods disclosed herein can be referred to as “fresh-flower dry-ice kief” (FDK) or Kryo-Kief. In some embodiments, the isolated trichomes have a biochemical profile of cannabinoids or terpenoids that is substantially similar to the biochemical profile of cannabinoids or terpenoids of the native plant material. The biochemical profile can include 1, 2, 3, 4, 5, 6, 7, 8, 9, 10 or more cannabinoids and/or terpenoids. “Substantially similar” as used herein can be defined as having at least 50% similarity to the profile found in the native starting plant material. In some embodiments, the isolated trichomes can have 50%, 55%, 60%, 65%, 70%, 75%, 80%, 85%, 90% or more percent similarity to the profile of the native starting plant material. The percentage of the profile can be determined by weight, concentration, ratios and/or number of distinct chemical components.

In some embodiments, the isolated trichomes have yields of 1-10% of the original flower mass. For example, the isolated trichomes can have a yield of 1%, 2%, 3%, 4%, 5%, 6%, 7%, 8%, 9%, 10%, or more of mass. In some embodiments, the increased yield of isolated trichomes as a function of original fresh (wet) weight of flower, as compared with the yield of trichome mass obtained from dried flower or other comparison processes (when adjusted to take into account the original fresh weight of the dried flower) can be 2×, 3×, 4×, 5×, 6×, or more. Thus, the invention provides a significant improvement in efficiency of recovery of trichomes, and their contents, as compared with comparison processes using dried flower. As used herein, a “comparison process” can include any traditional process such as air drying.

### Extraction

In some embodiments, the method or system includes extraction of plant compounds from the kief. The extraction can include supercritical CO₂ extraction techniques, cold ethanol extraction techniques, steam distillation, and/or any other extraction technique known in the art. Some techniques are discussed in Sarma, N. D., et al (2020). Cannabis Inflorescence for Medical Purposes: USP Considerations for Quality Attributes. J Nat Prod, 83(4), 1334-1351. doi:10.102¹/ₐcs.jnatprod.9b01200, which is incorporated herein by reference in its entirety.

In some embodiments, supercritical CO₂ extraction techniques are employed. See, e.g., Russo, E. B. (2003). Introduction: Cannabis: From pariah to prescription. Journal of Cannabis Therapeutics, 3(3-4), 1-29, which is incorporated herein by reference in its entirety. Illustrative parameters used for CO₂ extraction in one or more embodiments can include for example: CO₂ pressure in the range of 1000 psi to 1300 psi, forming a supercritical fluid; temperature between 80° F. and 100° F.; and elapsed time of exposing the Cannabis plant material to the supercritical CO₂ in the range of 15 minutes to 6 hours. After CO₂ extraction and removal of CO₂ (for example by reducing pressure to allow the CO₂ to evaporate), the terpene oil and terpene hydrosol may be filtered at a temperature between —80° F. and 40° F., using a filter with pore size greater than 0.25 micron.

In some embodiments, cold ethanol extraction techniques are employed. The resulting material can be roto-vaped to purge ethanol for concentrate production, vaporization, etc. Illustrative parameters used for cold ethanol extraction and ethanol recovery in one or more embodiments may include for example: flushing of residual plant material with cold ethanol at a temperature of 30° F. or below; and distilling the ethanol oil solution at a temperature between 120° F. and 165° F. under a vacuum between 10 inches Hg and 25 inches Hg. Recovered ethanol can be optionally reused for subsequent washing of a second batch of material.

In some embodiments, steam distillation extraction techniques are employed. See, e.g., Potter, D. J. (2009). The propagation, characterisation and optimisation of Cannabis sativa L. as a phytopharmaceutical. (PhD). King's College, London, which is incorporated herein by reference in its entirety. Distilling can be performed in one or more embodiments under vacuum with a pressure at or below 5 ton. Cannabinoid distillates can be obtained at a temperature of between 157° C. and 230° C. In one or more embodiments, distillation can also yield terpene distillates, for example at a temperature between 140° C. and 157° C. Distillation of these products may be performed multiple times to increase concentration or purity, followed by blending of terpene oil with the cannabinoid distillates.

### Decarboxylation

In some embodiments, the method can include decarboxylation of the extracts.

The decarboxylation can be done using an extractor such as a Soxhlet extractor. See, e.g., Pegoraro, C. N., Nutter, D., Thevenon, M., & Ramirez, C. L. (2019). Chemical profiles of Cannabis sativa medicinal oil using different extraction and concentration methods. Nat Prod Res, 1-4. doi:10.1080/14786419.2019.1663515, which is incorporated herein by reference in its entirety. This can be a closed system that decarboxylates acid cannabinoids to yield THC, CBD, CBC, CBG, THCV, CBDV and other neutral phytocannabinoids, while retaining terpenoids

### Extracted Material

In some embodiments, the extracted material can be substantially free of chlorophyll, phytol or extraneous lipid components. Substantially free can be defined as a reduction of at least 50%, 60%, 70%, 80%, 90%, 99%, or more of the chlorophyll, phytol or extraneous lipid components that was present prior to extraction. The combination of alternative secondary processing techniques (e.g., extraction techniques) after FDK isolation of trichomes can provide phytocannabinoid and cannabis terpenoid fractions of high quality and purity, with less extraneous material such as chlorophyll, phytol or extraneous lipid components compared to fractions isolated by traditional methods. In some embodiments, the extracted material shows purer color and less extraneous vegetative matter, such as non-glandular trichomes compared to samples isolated by traditional methods. FDK isolation as used herein can refer to the methods disclosed herein to prepare the plant material for extraction. In some embodiments, purity can be defined a useful phytochemicals vs total mass. Useful phytochemicals can include any desirable API, cannabinoid and/or terpenoid. In some embodiments, purity can be defined as percent cannabinoids plus terpenoids of total mass. In some embodiments, the extracted material can comprise a 0.5×, 1×, 2×, 3×, 4×, or 5× increase in concentration compared to fresh flower. Color of trichome collection can also be used to determine purity of the isolated material, e.g., can distinguish purer FDK from dry sifted material that has more debris and chlorophyll contamination. Examples of analysis of samples and other information can be found for example in Ross S A, ElSohly M A. The volatile oil composition of fresh and air-dried buds of Cannabis sativa. J Nat Prod. 1996 Jan;59(1):49-51. doi: 10.1021/np960004a. PMID: 8984153, which is incorporated by reference in its entirety herein.

### Lipid Nanoparticles

In some embodiments, the products produced from any of the methods disclosed herein can be incorporated into lipid nanoparticles. For example, the isolated trichomes can be dissolved in ethanol and the resulting material can serve as a base for assembly of lipid nanoparticles. As a non-limiting example, these can be used intravenously to treat cancer, as in Joshi M D, Müller R H. Lipid nanoparticles for parenteral delivery of actives. Eur J Pharm Biopharm. 2009 February; 71(2):161-72. doi: 10.1016/j.ejpb.2008.09.003. Epub 2008 Sep. 13. PMID: 18824097, which is hereby fully incorporated by reference herein. This will concentrate the extracted material in areas of higher blood flow, preferentially to the tumor itself.

### Other Processing

In some embodiments, any of the methods disclosed herein can be combined with other techniques (e.g., nanofiltration, centrifugal chromatography) to produce any desired combination of acid and neutral phytocannabinoids and cannabis terpenoids that can be tailored to specific therapeutic indications or industrial applications.

### System

Some methods of the invention relate to a system using any of the methods disclosed herein, wherein the system combines process steps with apparatus adapted to carry out the process steps.

### Advantages of the Invention

Non-limiting advantages of the invention can include one or more of the following:
1. There is essentially no undesired decarboxylation of acid cannabinoids that can occur by other isolation techniques, such as drying.
2. Terpenoid content, especially monoterpenoid content, is substantially retained in FDK. Most other approaches have much greater losses of monoterpenoids akin to that reported by Ross et al.
3. The FDK can allow use as-is for pharmaceutical, medical or recreational purposes, or secondary processing to decarboxylate the acid cannabinoids to neutral cannabinoids.
4. FDK is substantially free of extraneous chlorophyll and other phytochemicals that are not necessary or beneficial for pharmacological purposes.
5. Prior uses of dry ice have been predominantly with dried flower, sugar leaves, “shake” or other waste materials as opposed to the process of FDK where the entire point is to process freshly harvested inflorescences immediately.
6. Other applications typically employ granular dry ice to pulverize the flower and separate trichomes. The undesirable consequence is that much more leaf and other particulates are produced.
7. Water hash is a popular technique but leaves a large mass of wet plant matter that is either undesirable or requires massive processing to extract anything useful. The wastewater from this process is a pollutant. The contrast to FDK is striking. The remaining “frozen sifted flower” retains useful phytochemicals and can undergo secondary processing with no resultant water or other pollutants. Thus, a high value product and secondary product are both available.

### EXAMPLES

### Example 1

Experiments were done to compare various techniques with methods disclosed herein.

Cannabis inflorescences were freshly harvested and manicured by hand to remove stems and “sugar leaves.” Starting weight was measured. Flower material was then treated by placement in a metal casserole dish on a bed of dry ice within a polyethylene cooler, and a metal tray placed above with an additional bed of dry ice. The cooler's drain plug was opened to allow full egress of CO₂ and water vapor and allowing laminar flow of vapor over and through cannabis inflorescences to maximize penetration and lyophilization effects.

At the end of dry ice treatment, material was quickly reweighed, and placed in the Pollinator drum. The Pollinator unit was placed inside a chest freezer (0° F./−18 C.°) and run at 33 RPM.

After treatment, inflorescences were reweighed, and trichome material (FDK) collected for analysis. Two Pollinator drums were employed and cleaned between used with brushing and ethanol treatment.

For comparison, equivalent starting weights of each chemovar were dried to an approximate 10% moisture content and run through the Pollinator machine analogously to the fresh/frozen materials. All samples were sent for cannabinoid and terpenoid analysis. This was undertaken at Lightscale Labs, Portland, OR https://lightscale.com/).

Biochemical profiles and visual differences of the following groups were compared:
Fresh flower: cannabis inflorescences, separated by hand with scissors and with “sugar leaves” removed from four cannabis chemovars.
Frozen sifted flower: flower material after dry ice treatment
Material produced by invention (“FDK”): cannabis trichomes after dry ice and Pollinator sifting treatment
Dried flower: cannabis inflorescence after air drying
Dried sifted flower: dried flower after Pollinator sifting treatment
Dried Kief: trichome material after drying and sifting

### Example 2

Experiments as described in Example 1 were conducted in the Cannabis variety ‘Doug Fir’. 100 g of plant material was treated with dry ice for 1 hour and processed in the Pollinator for 5 minutes. Biochemical profiles of the isolated material were determined and the following results were obtained, all in percentage by weight:

| | | Frozen | | | Dried | |
|---|---|---|---|---|---|---|
| | Fresh | Sifted | | Dried | Sifted | Dried |
| | Flower | Flower | FDK | Flower | Flower | Kief |
| Cb. Total | 24.8 | 17.4 | 60.7 | 22.1 | 20.9 | 58.8 |
| THCA | 24.1 | 16.9 | 57.4 | 22 | 20.8 | 56.3 |
| THC | 0.583 | 0.499 | 1.05 | 0 | 0 | 1.08 |
| CBDA | 0 | 0 | 0.239 | 0 | 0 | 0.0934 |
| CBD | 0 | 0 | 0 | 0 | 0 | 0 |

| | | Frozen | | | Dried | |
|---|---|---|---|---|---|---|
| | Fresh | Sifted | | Dried | Sifted | Dried |
| | Flower | Flower | FDK | Flower | Flower | Kief |
| Terp. Total | 2.62 | 1.38 | 6.81 | 4.03 | 4.19 | 7.49 |
| Myrcene | 0.0945 | 0.103 | 0.0518 | 0.221 | 0.324 | 0.583 |
| Alpha- | 0.454 | 0.268 | 0.879 | 0.936 | 0.842 | 1.17 |
| pinene | | | | | | |
| Limonene | 0.324 | 0.212 | 1 | 0.507 | 0.605 | 0.758 |
| Caryophyllene | 0.264 | 0.243 | 0.791 | 0.647 | 0.615 | 0.728 |
| Beta- | 0.245 | 0.138 | 0.708 | 0.53 | 0.512 | 0.694 |
| pinene | | | | | | |
| Sabinene | 0.212 | 0.119 | 0.758 | 0.483 | 0.501 | 0.748 |

Visual differences are depicted in FIG. 2.

### Example 3

Experiments as described in Example 1 were conducted in the Cannabis variety ‘Astral Works’. 100 g of plant material was treated with dry ice for 1 hour and processed in the Pollinator for 5 minutes. Biochemical profiles of the isolated material were determined and the following results were obtained, all in percentage by weight:

| | | Frozen | | | Dried | |
|---|---|---|---|---|---|---|
| | Fresh | Sifted | | Dried | Sifted | Dried |
| | Flower | Flower | FDK | Flower | Flower | Kief |
| Cb. Total | 11.8 | 9.4 | 36.7 | 11.5 | 12 | 25.3 |
| THCA | 4.51 | 3.67 | 13.6 | 4.43 | 4.54 | 10 |
| THC | 0 | 0 | 0.34 | 0 | 0 | 0 |
| CBDA | 7.26 | 5.67 | 20.4 | 7.05 | 7.43 | 14.9 |
| CBD | 0 | 0 | 0.136 | 0 | 0 | 0 |

| | | Frozen | | | Dried | |
|---|---|---|---|---|---|---|
| | Fresh | Sifted | | Dried | Sifted | Dried |
| | Flower | Flower | FDK | Flower | Flower | Kief |
| Terp. Total | 1.59 | 0.94 | 4.22 | 1.68 | 2.15 | 2.07 |
| Terpinolene | 0.368 | 0.219 | 0.684 | 0.313 | 0.489 | 0.245 |
| Myrcene | 0.362 | 0.165 | 0.642 | 0.253 | 0.485 | 0.251 |
| Beta- | 0.202 | 0.106 | 0.483 | 0.157 | 0.276 | 0.167 |
| ocimene | | | | | | |
| Guiaiol | 0.127 | 0.111 | 0.195 | 0.13 | 0.113 | 0.152 |
| Caryophyllene | 0.0874 | 0.0704 | 0.442 | 0.163 | 0.144 | 0.237 |
| Alpha- | 0.0442 | 0.0173 | 0.321 | 0.0822 | 0.0906 | 0.19 |
| Pinene | | | | | | |

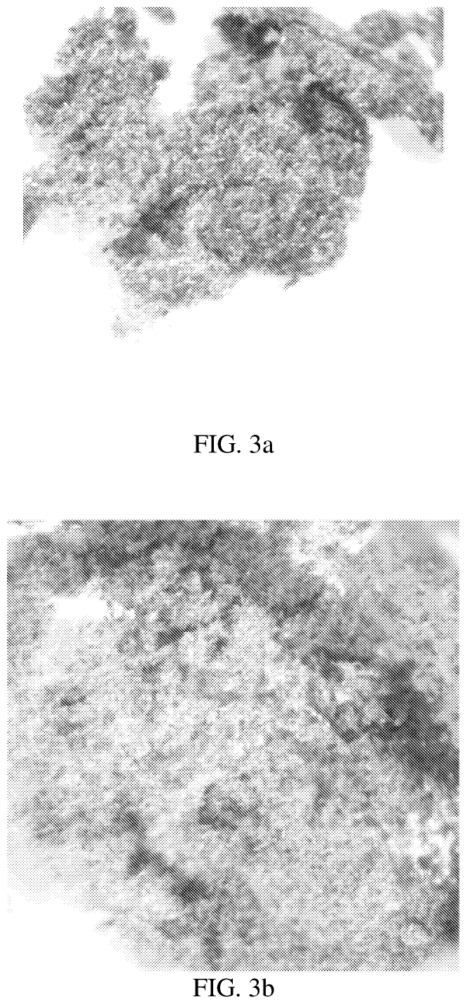

Yield was 0.1 g=0.1% of fresh wet weight. Visual differences are depcited in FIG. 3.

### Example 4

Experiments as described in Example 1 were conducted in the Cannabis variety ‘Tangie Biscotti’. 200 g of plant material was treated with dry ice for 48 hours and processed in the Pollinator for 20 minutes. Biochemical profiles of the isolated material were determined and the following results were obtained, all in percentage by weight:

| | | Frozen | | | Dried | |
|---|---|---|---|---|---|---|
| | Fresh | Sifted | | Dried | Sifted | Dried |
| | Flower | Flower | FDK | Flower | Flower | Kief |
| Cb. Total | 11.5 | 17.9 | 58.5 | 15.2 | 15.5 | 51 |
| THCA | 11.5 | 17.4 | 56.3 | 15 | 15.3 | 49.2 |
| THC | 0 | 0 | 1.05 | 0 | 0 | 0 |
| CBDA | 0 | 0 | 0.0129 | 0 | 0 | 0.572 |
| CBD | 0 | 0 | 0 | 0 | 0 | 0 |
| CBGA | 0 | 0.484 | 1.13 | 0.262 | 0.211 | 1.18 |
| CBG | 0 | 0 | 0 | 0 | 0 | 0 |

| | | Frozen | | | Dried | |
|---|---|---|---|---|---|---|
| | Fresh | Sifted | | Dried | Sifted | Dried |
| | Flower | Flower | FDK | Flower | Flower | Kief |
| Terp. Total | 1.38 | 0.968 | 2.87 | 1.2 | 1.12 | 2.49 |
| Selinadiene | 0.64 | 0.647 | 0.0167 | 0.0115 | 0.103 | 0.0138 |
| Caryophyllene | 0.364 | 0 | 0.797 | 0.532 | 0.486 | 0.656 |
| Humulene | 0.104 | 0.0712 | 0.218 | 0.136 | 0.128 | 0.161 |
| Myrcene | 0.0958 | 0.0551 | 0.713 | 0.143 | 0.146 | 0.67 |
| Bisabolol | 0.0572 | 0.044 | 0.0553 | 0.029 | 0.0335 | 0.0273 |
| Limonene | 0.0478 | 0.0524 | 0.0553 | 0.114 | 0.0971 | 0.404 |
| Linalool | 0.0495 | 0.0598 | 0.299 | 0.11 | 0.115 | 0.261 |
| Alpha-pinene | 0.0035 | 0.0046 | 0.0857 | 0.0106 | 0.00866 | 0.0526 |

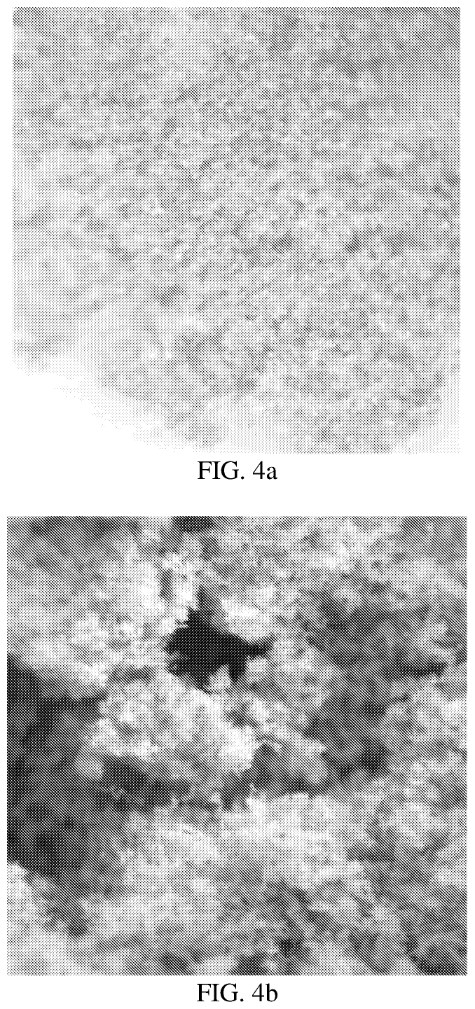

FDK appears extremely clean with rare green leaf flecks in comparison to dried kief (FIG. 4). Yield of 8.12 g from 200 g flower=4.06% of fresh wet flower weight. The Dried Kief group had a Yield of 1.68 g from 23.25 g dried flower=7% of dry weight, which converts to 0.84% of original wet weight. Thus, the difference in yield is a factor of 4.06/0.84=4.83× improved yield using the approach of the present invention.

### Example 5

Experiments as described in Example 1 were conducted in the Cannabis variety ‘Ursa Major’. 200 g of plant material was treated with dry ice for 48 hours and processed in the Pollinator for 20 minutes. Biochemical profiles of the isolated material were determined and the following results were obtained, all in percentage by weight:

| | | Frozen | | | Dried | |
|---|---|---|---|---|---|---|
| | Fresh | Sifted | | Dried | Sifted | Dried |
| | Flower | Flower | FDK | Flower | Flower | Kief |
| Cb. Total | 29.6 | 36.7 | 57.1 | 24.1 | 36 | 48.5 |
| THCA | 27.2 | 34.3 | 53.9 | 23.7 | 33.8 | 45.6 |
| THC | 0 | 0 | 0 | 0 | 0 | 0.276 |
| CBDA | 0 | 0 | 0.0242 | 0 | 0.0111 | 0.0494 |
| CBD | 0 | 0 | 0 | 0 | 0 | 0 |
| CBGA | 1.83 | 2.48 | 3.14 | 0.418 | 2.18 | 2.57 |
| CBG | 0 | 0 | 0 | 0 | 0 | 0 |

| | | Frozen | | | Dried | |
|---|---|---|---|---|---|---|
| | Fresh | Sifted | | Dried | Sifted | Dried |
| | Flower | Flower | FDK | Flower | Flower | Kief |
| Terp. Total | 1.41 | 0.956 | 2.73 | 2.3 | 2.18 | 2.41 |
| Selinadiene | 0.03 | 0.0376 | 0.0341 | 0.0359 | 0.0438 | 0.0368 |
| Caryophyllene | 0.807 | 0.472 | 1.38 | 1.23 | 1.34 | 1.15 |
| Humulene | 0.223 | 0.122 | 0.366 | 0.303 | 0.322 | 0.286 |
| Myrcene | 0.00922 | 0.0202 | 0.193 | 0.199 | 0.0277 | 0.136 |
| Bisabolol | 0.0195 | 0.00915 | 0.0414 | 0.0262 | 0.0215 | 0.0258 |
| Limonene | 0.0356 | 0.044 | 0.182 | 0.159 | 0.055 | 0.192 |
| Linalool | 0.248 | 0.204 | 0.444 | 0.2 | 0.315 | 0.437 |
| Alpha-pinene | 0.00405 | 0.00589 | 0.016 | 0.012 | 0.00506 | 0.0241 |

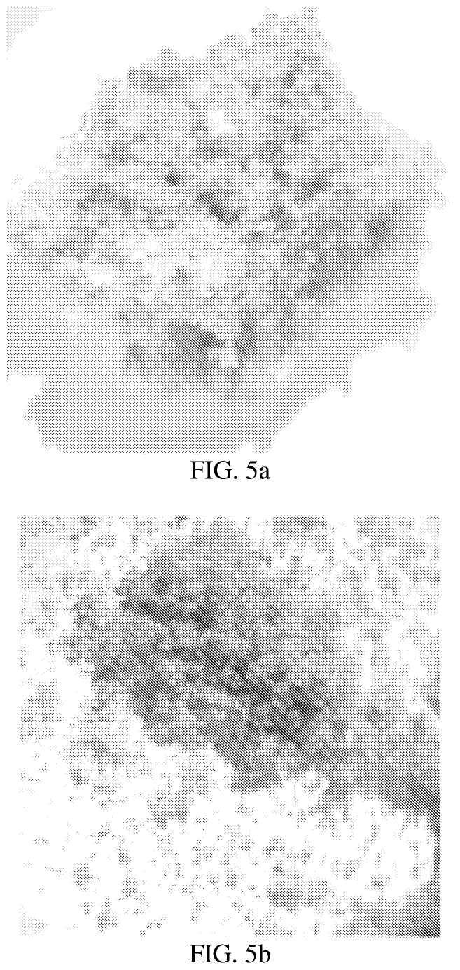

FDK appears much purer, with rare leaf flecks in comparison to dried kief (FIG. 5). Yield from 200 g sample wet weight in a first run=2.51 g or 1.26%. In a second run, 31.6 g were derived from 454 g=6.96% yield. In the dried kief sample, there was a yield of 2.66 g from 55.5 g dry flower=4.79%, or 1.33% of original wet weight. It was noted that in the first run, there was a problem in that the flower material was not shielded from above, allowing dry ice particles to rain down and induce water ice condensation. This was prevented in the second run, which is likely a more accurate figure on yield. Comparison of the percentage of yield as a function of original wet weight is 6.96/1.33=5.23× improved yield using the approach of the present invention as compared with more traditional approaches.

### Example 6

Experiments are done to compare various techniques with methods disclosed herein. Cannabis inflorescences are freshly harvested and manicured by hand to remove stems and “sugar leaves.” Starting weight is measured. Flower material is treated in two or more of the following groups as described in Example 1.
Fresh flower
Frozen sifted flower
Material produced by invention (FDK)
Dried flower
Dried sifted flower
Dried Kief

Biochemical profiles and visual differences of the treatment groups are compared.

### Example 7

Experiments as described in Example 6 are conducted in using a specific Cannabis variety. 100 g-5 kg of plant material is harvested expeditiously from growing plants and treated with dry ice and processed in the Pollinator (or similar device) for 24-48 hours. Analyses of the samples show that FDK samples have increased preservation of terpenoid content and acid cannabinoid content as they appear in fresh flower compared to Dried Kief samples of the same variety.

### Example 8

Experiments as described in Example 6 are conducted in using a specific a Cannabis variety. 100 g-5 kg of plant material is harvested expeditiously from growing plants and treated with dry ice and processed in the Pollinator (or similar device) for 24-48 hours. Colorimetric analyses of the trichomes show that FDK samples have a more similar color to trichomes of fresh flower compared to trichomes of Dried Kief samples of the same variety.

### Example 9

Experiments as described in Example 6 are conducted in a Cannabis variety. 100 g-5 kg of plant material is harvested expeditiously from growing plants and treated with dry ice and processed in the Pollinator (or similar device) for 24-48 hours. Analyses of the samples show that FDK samples have less extraneous vegetative matter compared to Dried Kief samples. Extraneous vegetative matter includes non-glandular trichomes and leaf or stem particles and/or the like.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A method of preparing a Cannabis plant material for extraction of a compound comprising:
a. obtaining fresh plant material;
b. flash freezing the plant material in a container containing a cooling material for a period of time; and
c. dry sifting the plant material in a cold environment to isolate trichomes; wherein the isolated trichomes comprise a biochemical profile of cannabinoids or terpenoids that is substantially similar to the biochemical profile of the fresh plant material.

2. The method of claim 1, wherein the cooling material is dry ice.

3. The method of claim 1, wherein the time between step a and b is less than 5 minutes.

4. The method of claim 1, wherein the period of time in step b is at least 10 minutes.

5. The method of claim 1, wherein the dry sifting step comprises placing the frozen plant material in a rotating drum with perforations.

6. The method of claim 5, wherein the isolated trichomes are collected in a tray underneath the drum.

7. The method of claim 1, wherein no solvents are used.

8. The method of claim 1, wherein the relative yield of isolated trichomes is at least 2× greater than a yield of isolated trichomes from a comparison process, wherein the comparison process begins with an equivalent amount of starting material and comprises drying the plant material prior to isolation of trichomes.

9. The method of claim 8, wherein the relative yield of isolated trichomes is at least 3× greater than the yield of isolated trichomes from the comparison process.

10. The method of claim 9, wherein the relative yield of isolated trichomes is at least 4× greater than the yield of isolated trichomes from the comparison process.

11. (canceled)

12. A method of extracting a compound from a Cannabis plant using the method of claim 1 and further comprising:
d. extracting compounds from trichomes;
e. optionally, decarboxylating the compounds; wherein the isolated compound comprises at least 0.5× more purity compared to fresh flower, wherein the purity is defined by percent total cannabinoids and terpenoids of the total mass.

13. The method of claim 9, wherein the extraction step comprises use of an extraction medium or process selected from at least one of supercritical CO2, cold ethanol, and steam distillation.

14. A lipid nanoparticle preparation produced from an isolated trichome produced by the method according to claim 1.
